(19) United States
(12) Patent Application Publication
(10) Pub. No.: US 2004/0089297 A1
(43) Pub. Date: May 13, 2004

(54) Absorber arrangement for anesthesia apparatus

(75) Inventor: Petter Videbrink, Upplands Vasby (SE)

Correspondence Address: SCHIFF HARDIN, LLP, PATENT DEPARTMENT, 6600 SEARS TOWER, CHICAGO, IL, 60606-6473, US

(73) Assignee: Maquet Critical Care AB

(21) Appl. No.: 10/700,695

(22) Filed: Nov. 4, 2003

(30) Foreign Application Priority Data
Nov. 13, 2002 (SE) 0203342-1

(51) Int. Cl. A61M 15/00; A61M 16/10

(52) U.S. Cl. 128/203.12; 128/910

(57) ABSTRACT

An absorber arrangement for an anesthesia apparatus has a docking unit and a cassette unit, removably connected to the docking unit, the docking unit having a valve that shuts off flow paths in the absorber arrangement when the cassette unit is detached from the docking unit. Increased functionality with fewer components is made possible by the valve being formed by a shunt valve adapted to by-pass the flow paths in the absorber arrangement dependent on the position of the cassette unit in the docking unit and the cassette unit is freely positionable between a first position and a second position, the bypass being 100% for the cassette unit in the first position and 0% for the cassette unit in the second position.

### BACKGROUND OF THE INVENTION

1. Field of the Invention

The present invention relates to an absorber arrangement of the type wherein a cassette unit is removably connected to a docking unit, and wherein the docking unit includes a valve that shuts off flow paths in the absorber arrangement when the cassette unit is detached from the docking unit.

2. Description of the Prior Art

Anesthesia apparatuses can be designed with many different types of breathing circuits that in principle can be categorized more or less as open and closed. At one extreme there is the completely open system in which all of the breathing gas that comes from a patient during exhalation is evacuated. This results in a large consumption of expensive anesthetic and is therefore, seldom used.

At the other extreme there is the completely closed system. In this system carbon dioxide is removed from the exhaled breathing gas, which is then re-supplied to the patient with the addition of gases to compensate for those taken up by the patient's body via the lungs, primarily oxygen. The closed system uses substantially smaller quantities of the expensive anesthetic but requires a greater degree of control and monitoring.

The majority of “closed” systems therefore utilize greater gas consumption than is necessary, in order to make the control and monitoring easier.

The removal of carbon dioxide is done in absorber arrangements connected to the flow paths of the breathing circuits, for example as described in PCT Application WO 92/05826. The absorber arrangement includes a carbon dioxide absorber that, when full, must be exchanged. The absorber arrangement therefore normally has a docking unit and a cassette unit, the latter being exchangeable. In order to prevent the escape of breathing gas from the breathing circuit as the exchange of the cassette section takes place, a valve for shutting off flow paths within the absorber arrangement can be arranged in the docking section. This results in all of the exhaled gas being re-supplied to the patient during the time that it takes for the cassette unit to be exchanged. The valve may be designed so that it automatically shuts the flow paths when the cassette unit is disconnected.

Situations occur, however, in which a certain re-breathing of carbon-dioxide-containing gas is desirable. Supplying carbon dioxide after the absorber arrangement could, of course, solve this but an even simpler solution is to cause a portion of the exhaled breathing gas to bypass the absorber arrangement. Such a solution is described in Swedish Application 506 727 wherein a shunt valve is coupled in parallel with the absorber arrangement so as to be able to by-pass an adjustable amount of gas past the absorber arrangement. However, the system that is described in Swedish Application 506 727 lacks the possibility of shutting off the flow paths to the absorber arrangement when the cassette unit is changed.

### SUMMARY OF THE INVENTION

An object of the present invention is to provide an absorber arrangement which allows a change of the cassette unit to be simply and safely achieved without the risk of leakage and which also allows an adjustable amount of the exhaled breathing gas to bypass the absorber arrangement in a simple manner.

The above object is achieved in accordance with the present invention in an absorber arrangement for an anesthesia apparatus, having a docking unit and a cassette unit removably connected to the docking unit, the docking unit having a valve that shuts off flow paths to the absorber arrangement when the cassette unit is detached from the docking unit, and wherein the valve is formed as a shunt valve adapted to bypass the flow paths in the absorber arrangement dependent on the position of the cassette unit in the docking unit. The cassette unit is freely positionable between a first position and a second position, the bypass being 100% for the cassette unit in the first position and 0% for the cassette unit in the second position.

By designing the shut off valve in the docking unit so that it can be employed also as a shunt valve, regulated by the position of the cassette unit, a simple and robust system is achieved that with a minimum of components carries out many of the functions for which known systems employed separate components.

### DESCRIPTION OF THE DRAWINGS

FIG. 1 shows an absorber arrangement according to the invention.

FIG. 2 shows a valve of the inventive absorber arrangement.

### DESCRIPTION OF THE PREFERRED EMBODIMENTS

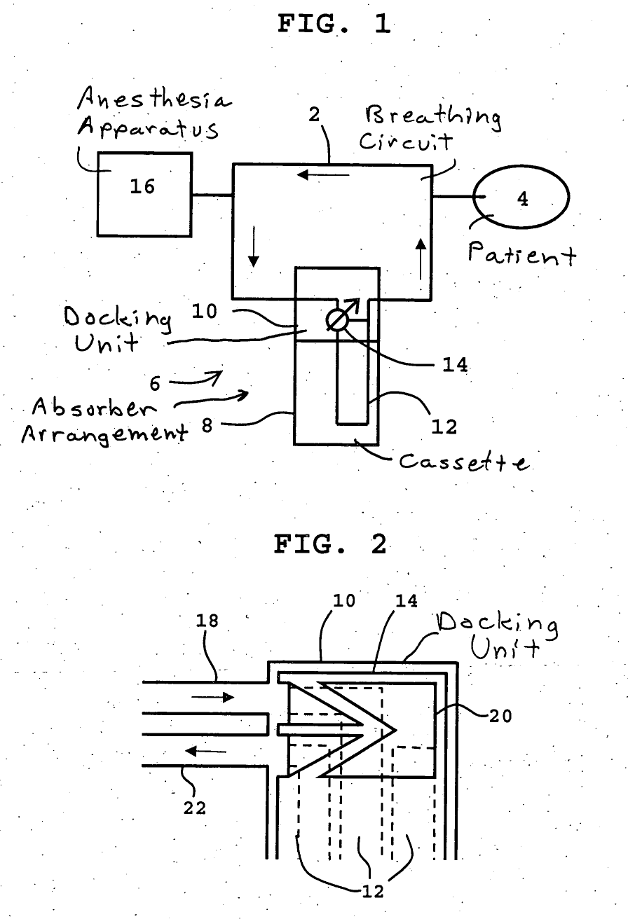

A breathing circuit 2 in an anesthetic apparatus 16 is shown in FIG. 1. The breathing circuit 2 is connected to a patient 4. Arrows indicate the flow direction for breathing gas within the breathing circuit 2.

An absorber arrangement 6 is arranged in the breathing circuit 2 to absorb carbon dioxide from the breathing gas before it is led to the patient 4. The absorber arrangement 6 has a cassette unit 8 and a docking unit 10. The cassette unit 8 can be disconnected from the docking unit 10 when the absorber (not shown) in the cassette unit 8 is saturated with carbon dioxide. In the present embodiment the cassette unit 8 is disconnected through a rotational movement of the cassette unit 8 about its own axis.

Flow paths 12 in the absorber arrangement 6 have been indicated schematically in FIG. 1. A valve 14 is arranged in the flow paths 12 between the docking unit 10 and the cassette unit 8. The valve 14 fulfils two primary functions.

The valve 14 functions in part as an automatic shut off valve when the cassette unit 8 is disconnected from the docking unit 10. All of the gas then flows directly to the breathing circuit 2.

The valve 14 also functions in part as a shunt valve. When the cassette unit 8 is connected to the docking unit 10 the valve 14 operates to gradually connect in the flow paths 12 until the cassette unit 8 has reached its end position (in this case a rotational end position). By locating the cassette unit 8 between these two positions a selectable portion of the breathing gas flow is caused to bypass coupled past the flow paths 12 in the cassette unit 8.

FIG. 2 shows the valve 14 in greater detail. The docking unit 10 is indicated whilst the cassette unit is omitted. The breathing gas is provided to the absorber arrangement via an inlet 18 and on to the valve 14. A shunt element 20 in the valve 14 regulates how much of the flow shall pass through the flow paths 12 and how much shall pass directly to an outlet 22. Adjustment is made dependent on the position (rotational position) of the shunt element 20. In one extreme position all of the gas is provided to the flow paths 12 and in the other extreme position all of the gas is provided directly to the outlet 22 (in this latter position the cassette unit can be disengaged from the docking unit 10).

As is shown in FIG. 2 the flow paths 12 are co-axially arranged, which eases the construction when a rotation of the shunt element 20 is employed to regulate the flow.

The same effect of the double functionality of closing and by-pass coupling may be achieved using other types of valves based on other directional movements, for example linear movement.

Although modifications and changes may be suggested by those skilled in the art, it is the intention of the inventor to embody within the patent warranted hereon all changes and modifications as reasonably and properly come within the scope of his contribution to the art.

I claim as my invention:

1. An absorber arrangement for an anesthesia apparatus, comprising:
a docking unit;
a cassette unit removably connected to the docking unit, said cassette unit, when connected to said docking unit, forming at least one flow path proceeding in said docking unit and said cassette unit and adapted to proceed through an anesthetic apparatus;
a valve in said docking unit operable to shut off said at least one flow path when said cassette unit is detached from said docking unit, and operable as a shunt valve to bypass said at least one flow path dependent on a position of said cassette unit in said docking unit; and
said cassette unit being freely positionable in said docking unit between a first position and a second position, said bypass being 100% when said cassette unit is in said first position and being 0% when said cassette unit is in said second position.

2. An absorber arrangement as claimed in claim 1 wherein said valve is rotatable relative to said at least one flow path and wherein said cassette unit is mechanically connected to said valve when said cassette is in said docking unit to selectively rotate said valve to change said bypass.

3. An absorber arrangement as claimed in claim 1 wherein said at least one flow path comprises coaxially disposed flow paths.
